(19) United States
(12) Patent Application Publication
(10) Pub. No.: US 2024/0325383 A1
(43) Pub. Date: Oct. 3, 2024

(54) Small Molecule Targeting of BRD4 for Treatment of COVID-19

(71) Applicant: Board of Regents, The University of Texas System, Austin, TX (US)

(72) Inventors: Alexander Bukreyev, Galveston, TX (US); Jia Zhou, Galveston, TX (US)

(73) Assignee: Board of Regents, The University of Texas System, Austin, TX (US)

(21) Appl. No.: 18/613,203

(22) Filed: Mar. 22, 2024

Related U.S. Application Data
(60) Provisional application No. 63/454,511, filed on Mar. 24, 2023.

(51) Int. Cl. A61K 31/496; A61K 31/573; A61K 31/675; A61K 45/06; A61P 11/02

(52) U.S. Cl. CPC A61K 31/496; A61K 31/573; A61K 31/675; A61K 45/06; A61P 11/02

(57) ABSTRACT

Embodiments are directed to molecules for the treatment of Coronavirus infection, including SARS-COV-2 infection. In certain aspects the small molecule is administered via inhalation and/or intranasal administration.

### RELATED APPLICATIONS

This application claims priority to U.S. Provisional Patent Application Ser. No. 63/454,511 filed Mar. 24, 2023 which is incorporated herein by reference in its entirety.

### STATEMENT REGARDING FEDERALLY FUNDED RESEARCH

None.

### BACKGROUND

In December 2019, an outbreak of a severe respiratory disease was first reported in the city of Wuhan, Hubei, China. The causative agent of this outbreak was identified as a novel coronavirus named severe acute respiratory syndrome coronavirus-2 (SARS-COV-2), causing COVID-19 (Zhu et al., 2020). The World Health Organization declared the outbreak a Public Health Emergency of International Concern on Jan. 30, 2020, and a pandemic on Mar. 11, 2020. It spread rapidly around the world, causing more than 261 million cases and 5.2 million deaths as of Nov. 30, 2021 (URL covid19.who.int). Since the last quarter of 2020, variant viruses have emerged in many parts of the world as a result of the high burden of infection and the adaptation of SARS-COV-2 to human cells under immune pressure (Andreano and Rappuoli, 2021; Subbarao, 2021).

There remains a need for improved treatment of respiratory viruses such as SARS-CoV-2.

### SUMMARY

One solution to the problem of SARS-COV-2 infection is the design, production, and administration of small molecules for the treatment of COVID19 or other Coronavirus infections.

The NFκB-BRD4 signaling pathway in airway epithelial cells mediates the acute inflammatory response to a variety of viruses and viral exposures. BRD4 inhibition was shown to completely block poly(I: C) and/or respiratory syncytial virus-induced inflammatory gene programs in vitro, and airway inflammation and neutrophil recruitment in vivo. Small molecule selective inhibitors of the BRD4 bromodomain were designed, synthesized, and demonstrated to be specific for a BRD4 domain (BD) with sub-micromolar affinity. Compounds described herein can be used to treat and/or ameliorate viral respiratory infections.

Certain embodiments are directed to methods of treating a patient infected with a coronavirus comprising administering to the patient a compound of Formula 1 or a pharmaceutically-acceptable salt thereof

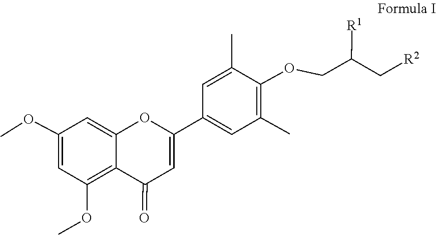

wherein R¹ is selected from hydrogen or hydroxyl; and R² is selected from a substituted or unsubstituted cycloalkyl, or a substituted or unsubstituted heterocycle. In certain aspects R¹ is hydrogen and R² is a substituted or unsubstituted heterocycle. In other aspects R² is a substituted heterocycle. The substituted heterocycle can be a substituted piperazine. In certain aspects the substituted piperazine is a methyl piperazine. In a further aspect the methyl piperazine is a 4 methyl piperazine. The coronavirus can be selected from the group consisting of SARS-COV, SARS-COV-2, and MERS-COV. In certain aspects the coronavirus is SARS-COV-2. The compound, or a pharmaceutically-acceptable salt thereof, can be administered by inhalation. In certain aspects the compound, or a pharmaceutically-acceptable salt thereof, is administered by nebulized inhalation. In certain aspects the compound, or a pharmaceutically-acceptable salt thereof, is administered once a day. The compound, or a pharmaceutically-acceptable salt thereof, can be administered at a higher loading dose on day 1 of administration followed by a lower dose on the following days. The methods can include administering one or more additional therapeutic agents or treatments to the patient. In certain aspects the patient receives standard of care co-treatment. The patient can be treated with corticosteroids. In certain aspects the patient can treated with dexamethasone. In certain aspects the patient can be treated with remdesivir. In certain aspects the compound of Formula 1, or a pharmaceutically-acceptable salt thereof, can be administered to the patient at a dose of about 1 mg to about 10 mg. In certain aspects the patient has mild to moderate COVID-19. In other aspects the patient has severe COVID-19. The patient can be at high risk for progressing to severe COVID-19 and/or hospitalization. In certain aspects the patient suffers from hypertension and/or diabetes.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

As used herein, the term “IC₅₀” refers to an inhibitory dose that results in 50% of the maximum response obtained.

The term half maximal effective concentration (EC₅₀) refers to the concentration of a drug that presents a response halfway between the baseline and maximum after some specified exposure time.

The terms “inhibiting,” “reducing,” or “ameliorating,” or any variation of these terms, when used in the claims and/or the specification includes any measurable decrease or complete inhibition to achieve a desired result.

As used herein, the term “patient” or “subject” refers to a living mammalian organism, such as a human, monkey, cow, sheep, goat, dogs, cat, mouse, rat, guinea pig, or species thereof. In certain embodiments, the patient or subject is a primate. Non-limiting examples of human subjects are adults, juveniles, and infants.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. The terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps) but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1A-1E. In vitro screening of 31 epigenetic inhibitors. Calu-3 cells seeded on transwell plates, treated with 10 μM compound 1 hour prior to infection with 0.6 PFU/cell rSARS-COV-2mNG, then treated daily for 2 days post-infection. (A) Fold change in CXCL10 expression produced with two-step real-time PCR assay and analyzed using comparative Ct relative quantitation method normalized to housekeeping gene B-Actin. Fold-change for three compounds of interest is indicated. Technical replicates were run in triplicate. *Note that YX0195 was toxic to the cell monolayer. (B) Log₁₀ viral titers produced with FFA. Three compounds of interest significantly reduce virus titer. P values for individual comparison of treated vs mock-treated samples are indicated. (limit of detection, 7FFU/mL). (C) Microscopy of three compounds of interest. Two compounds reduce CXCL10 expression (JMX0312 and ZL0969) one enhances CXCL10 expression (JMX0281). Bright field microscopy at 10× was used to assess general health of cell monolayer (toxicity of compounds). Fluorescent microscopy at 2× and 10× was used to assess GFP fluorescence associated with level of infection. (D) Chemical structures of three compounds of interest, salicylamide derivatives JMX0281 and JMX0312 and BRD4 inhibitor ZL0969. (E) TaqMan Human Cytokine Array heat map of mean cytokine expression pattern using two-step real-time PCR analyzed using comparative C, relative quantitation method and normalized to 4 housekeeping genes 18S, HPRTI, GAPDH, and GUSB.

FIG. 2A-2E. In vivo screening of 3 epigenetic inhibitors. Groups of BALB/c mice (n=4-5) treated with 20 mg/kg compound 2 days prior to challenge with 6×10⁴ PFU of the Beta variant (B.1.351) of SARS-COV-2 followed by daily treatments through day 9 post-infection. While this virus challenge dose is non-lethal in BALB/c mice, the South African variant produces infection. Animals were weighed daily and sacrificed 10 days post-infection. Viral load and changes in chemokine and cytokine expression are apparent in the lung by day 3 after infection with this strain of SARS-COV-2. (A) Body weight curves. *Note that one mouse in the Mock treated group died during challenge. (B) Viral load in lungs (Superior and Middle lobes). Each dot corresponds to an individual sample. Data represent mean±SEM of n=4-5 per group. Two-way ANOVA and a post hoc analysis comparing groups pairwise by time point. Dunnett's test was performed for multiple testing correction. Significance set at 0.05. P values indicated. (C) Representative lung tissue hematoxylin and eosin (H&E) staining at 20× magnification of lungs on day 2 post-challenge. Scale bar, 50 μm. (D) Histopathologic scoring of lung lesions on day 2 post-challenge. (E) Histopathologic scoring of lung lesions on day 10 post-challenge. D and E, data represent mean±SEM of n=4-5 per group. Two-way ANOVA was performed with significance set at 0.05. P values indicated.

FIG. 3. In vitro screening of 31 epigenetic inhibitors. Calu-3 cells seeded on transwell plates, treated with 10 μM compound 1 hour prior to infection with 0.6 PFU/cell rSARS-COV-2mNG, then treated daily for 2 days post-infection. Microscopy of thirty-one epigenetic inhibitors. 2× and 10× objectives used to capture images. Treatments are indicated to the left of each set of four columns. The first column in each set depicts cell monolayers treated with respective compound and infected with virus, images taken with 2× objective to visualize a large portion of the transwell surface area. The second column in each set depicts uninfected cell monolayers treated with respective compound to test for toxicity, images taken with 10× objective. Note that the last compound (YX0195) was shown to be very toxic to the cell monolayer. The last two columns in each set depict the cell monolayers treated with respective compound and infected with virus taken with 10× objective under bright field or fluorescent light. Bright field microscopy was used to assess general health of cell monolayer (toxicity of compounds). Fluorescent microscopy was used to assess GFP fluorescence associated with level of infection.

FIG. 4. Top ten compounds that show greatest reduction or increase in CXCL10 expression relative to mock-treated-infected Calu3 cells. Based on the data in FIG. 1A, which shows the fold changes in CXCL10 expression relative to uninfected cells, the fold-change relative to mock-treated-infected cells was calculated for these ten compounds. Actual fold changes are shown for JMX0281, JMX0312 and ZL0969.

FIG. 5. Toxicity testing of three compounds chosen for in vivo studies. Viability was calculated based on toxicity testing of three different concentrations of the three compounds in Calu-3 cells after 2 days exposure to treatments. Technical replicates of each sample were tested in triplicate. The average LC50 value for each compound is shown on the graph above the respective group. LC50 values were calculated based on linear equations generated in Excel from the average viability at each concentration.

FIG. 6. Average percent SARS overall alignment rate of three compounds from in vivo studies. Groups of BALB/c mice (n=4-5) treated with 20 mg/kg compound 2 days prior to challenge with 6×10⁴ PFU of the Beta variant (B.1.351) of SARS-COV-2 followed by daily treatments through day 9 post-infection. Mice were sacrificed on day 10 and RNA was prepared from the left lung. Next generation sequencing data of overall alignment rate with SARS-COV-2 confirms the titration data shown in FIG. 2B. Data represent mean±SEM of n=4-5 per group. Brown-Forsythe and Welch ANOVA tests of repeated measures one-way ANOVA data. Dunnett's test was performed for multiple testing correction. Significance set at 0.05. P values indicated.

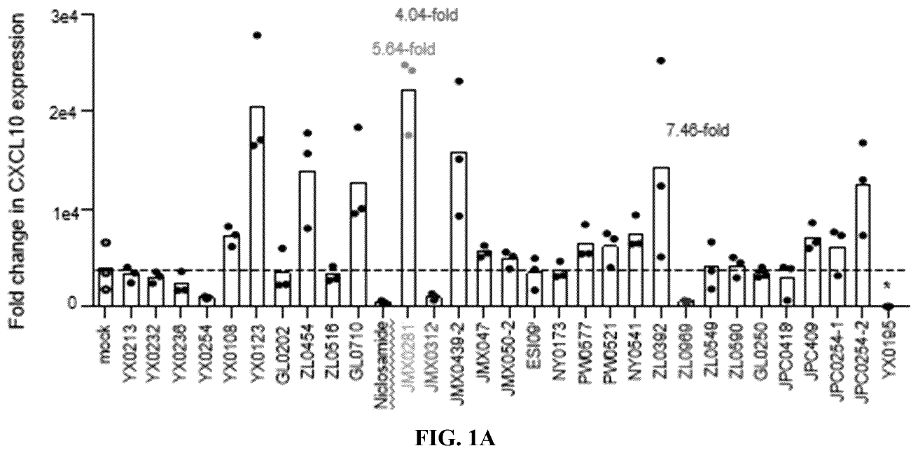

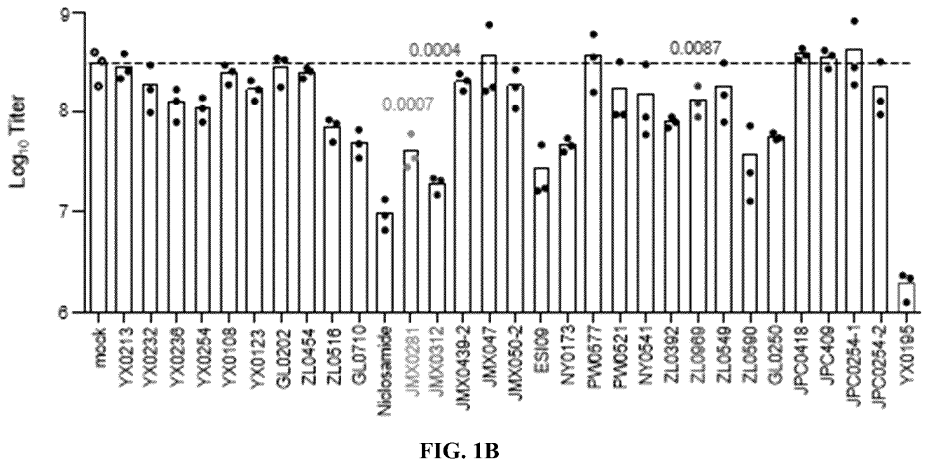

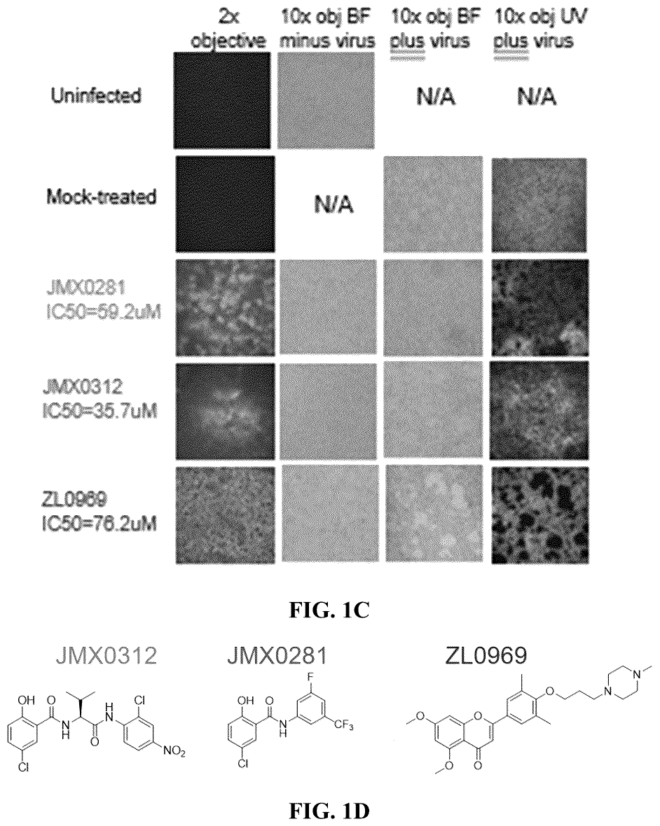

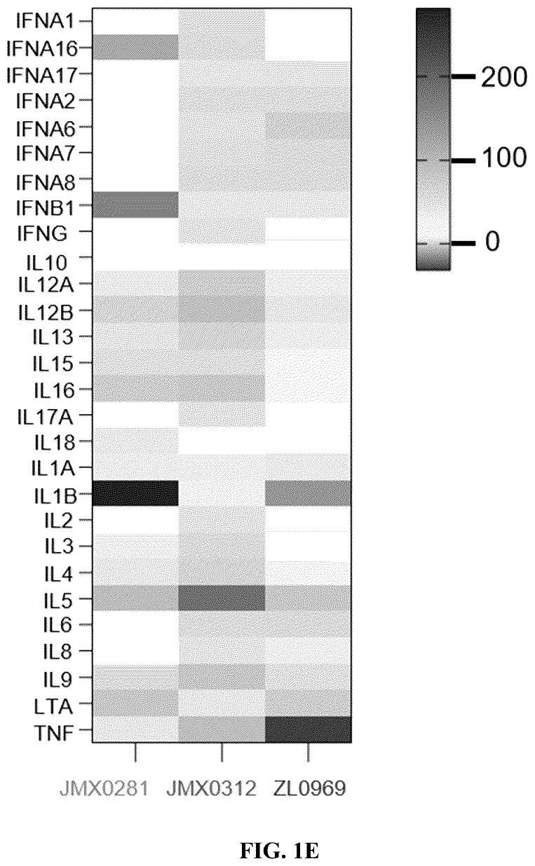

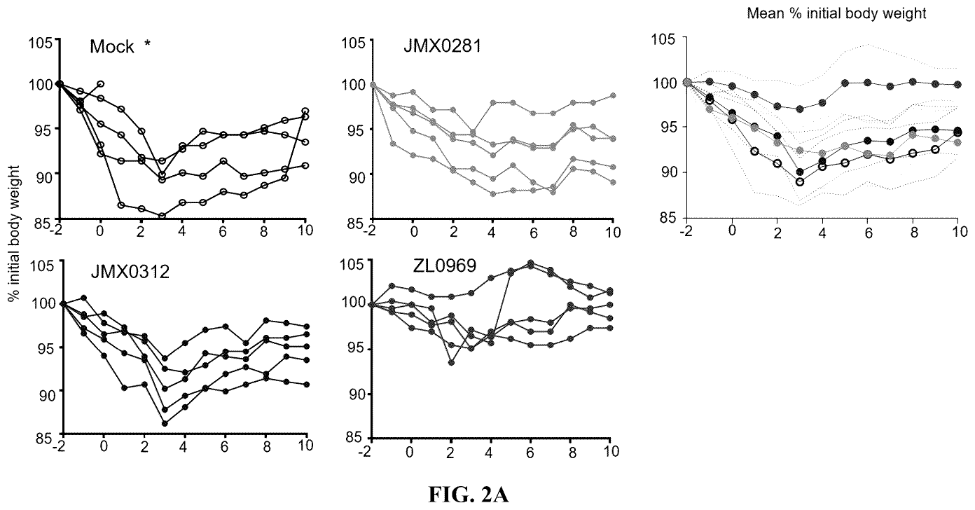

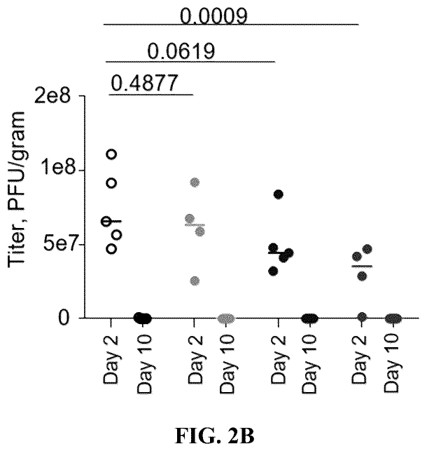

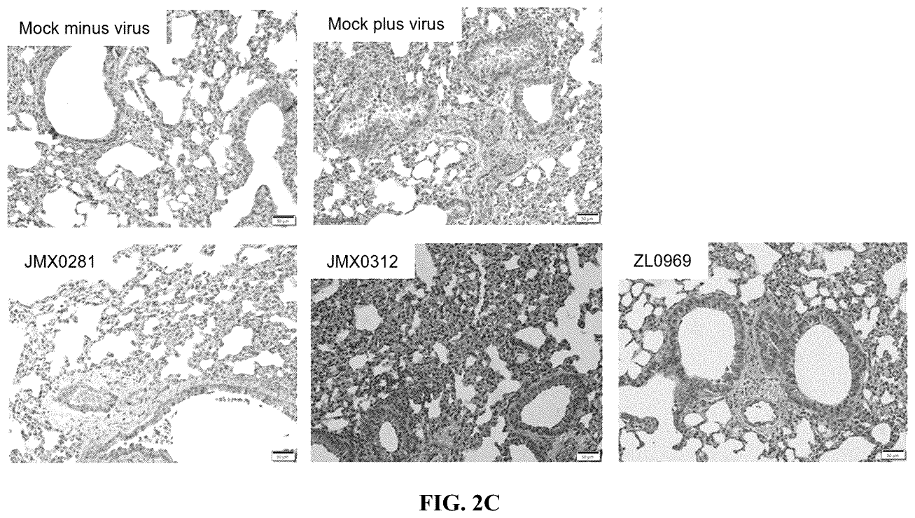

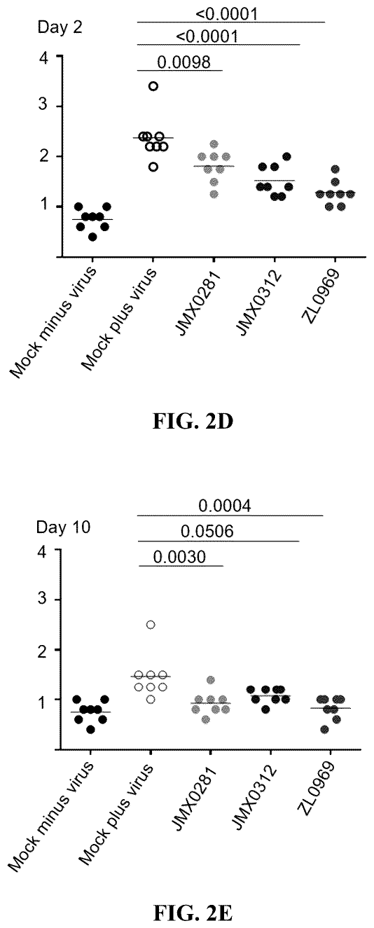

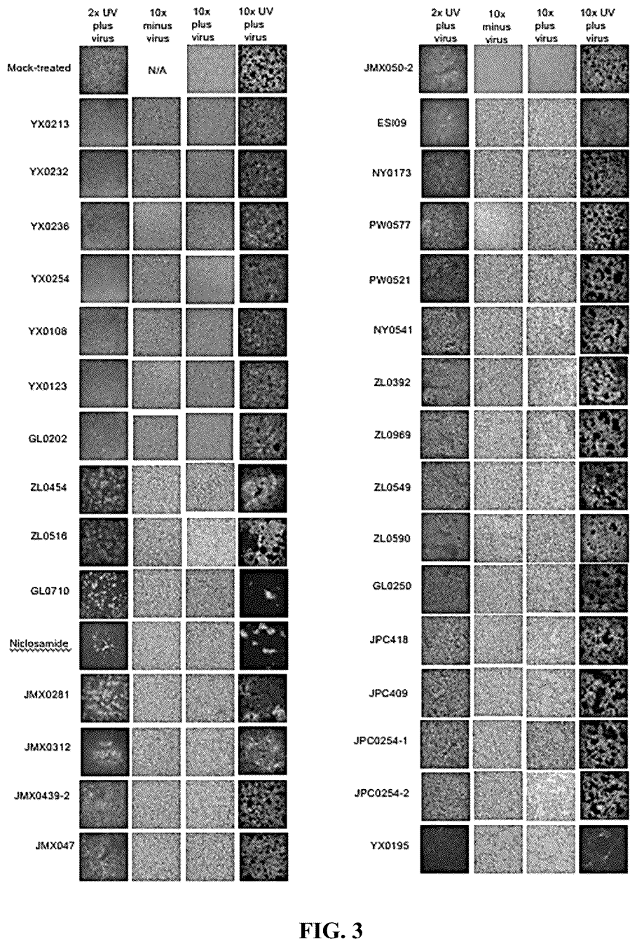

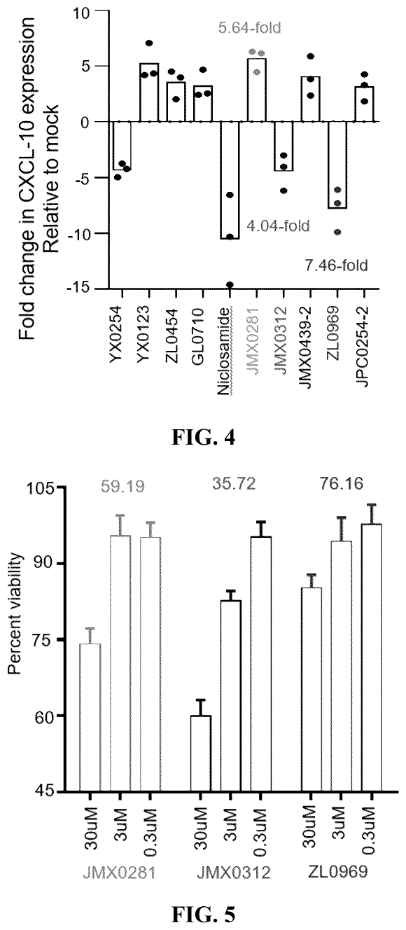

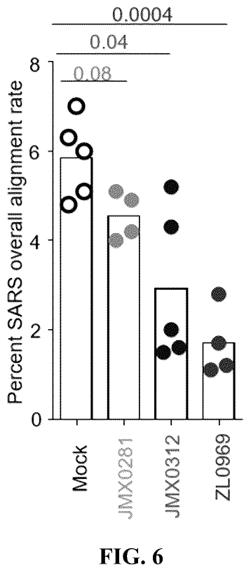

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be an example of that embodiment, and not intended to imply that the scope of the disclosure, including the claims, is limited to that embodiment.

Human coronavirus is a common respiratory pathogen and typically induces mild upper respiratory disease. The two highly pathogenic viruses, Severe Acute Respiratory Syndrome Coronavirus (SARS-COV-1) and Middle East Respiratory Syndrome-associated Coronavirus (MERS-COV), caused severe respiratory syndromes resulting in more than 10% and 35% mortality, respectively (Assiri et al., N Engl J Med., 2013, 369, 407-1). The recent emergence of Coronavirus Disease 2019 (COVID-19) and the resulting pandemic has created a global health care emergency. Similar to SARS-COV and MERS-COV, a subset of patients (about 16%) can develop a severe respiratory illness manifested by acute lung injury (ALI) leading to ICU admission (about 5%), respiratory failure (about 6.1%) and death (Wang et al., JAMA, 2020, 323, 11, 1061-69; Guan et al., N Engl J Med., 2020, 382, 1708-20; Huang et al., The Lancet, 2020. 395 (10223), 497-506; Chen et al., The Lancet, 2020, 395 (10223), 507-13). Accumulating evidence suggests that a subgroup of patients with COVID-19 might have a hyperinflammatory “cytokine storm” resulting in acute lung injury and acute respiratory distress syndrome (ARDS). This cytokine storm may also spill over into the systemic circulation and produce sepsis and ultimately, multi-organ dysfunction syndrome (Zhou et al., The Lancet, 2020, Vol. 395, Issue 10229, 1054-62). The dysregulated cytokine signaling that appears in COVID-19 is characterized by increased expression of interferons (IFNs), interleukins (ILs), and chemokines, resulting in ALI and associated mortality.

The bromodomain, a highly conserved motif of 110 amino acids, is found in proteins that interact with chromatin, such as histone acetylases, transcription factors and nucleosome remodeling complexes (Zeng and Zhou, FEBS Lett. 2002, 513:124-8). Bromodomain-Containing Protein 4 (BRD4), belonging to bromodomain and extra-terminal proteins (BET) family (BRD2, BRD3, BRD4 and BRDT), contains two bromodomains and functions as a chromatin “reader” that binds acetylated lysine in histones (Wu et al., Mol. Cell 2013, 49:843-57; Belkina and Denis, Nat. Rev. Cancer 2012, 12:465-77). It is an epigenetic reader and a critical regulator of transcription in many cell types. BRD4 plays an important role in the regulation of cell cycle control and transcription elongation mediated by interactions with P-TEFb (Jang et al., Mol. Cell 2005, 19:523-34). Oncogene BRD4-NUT has also been detected in tumor tissues (French et al., Cancer Res. 2003, 63:304-7). BRD4 is recently identified as a cancer therapeutic target for Basal-like breast cancer (Shi et al., Cancer cell 2014, 25:210-25), NUT midline carcinoma (NMC), acute myeloid leukemia, multiple myeloma, Burkitt's lymphoma and so on.

Meanwhile, BRD4 also has an essential role in the induction of inflammatory gene transcription. BRD4 is associated with nuclear factor-κB (NF-κB) signaling pathway via specific binding to acetylated RelA to stimulate NF-κB-dependent inflammatory response (Zou et al., Oncogene 2014, 33:2395-404). BRD4 is reported as a potential therapeutic target for patients with fibrotic complications (Ding et al., PNAS USA 2015, 112:15713-8). In addition, BRD4 competes with the HIV transactivator protein Tat for PTEFb binding to repress the Tat-mediated transactivation of the HIV promotor (Bisgrove et al., PNAS USA 2007, 104:13690-5). BRD4 inhibitors may efficiently reverse latent HIV (Li et al., Nucleic Acids Res. 2013, 41:277-87). Furthermore, BRD4 is crucial to neuronal function and mediates the transcriptional regulation underlying learning and memory. The loss of BRD4 function affects critical synaptic proteins, which results in memory deficits in mice but also decrease seizure susceptibility (Korb et al., Nat. Neurosci. 2015, 18:1464-73). Most recently, BRD4 was validated as an in vivo target for the treatment of pulmonary fibrosis associated with inflammation-coupled remodeling in chronic lung diseases (Tian et al., Am J Physiol Lung Cell Mol Physiol. 2016 Oct. 28). Targeting BRD4 represents a novel therapeutic method for a variety of different human diseases.

### I. BRD4 INHIBITORS

Certain embodiments are directed to BRD4 inhibiting compounds of Formula I, or a pharmaceutically acceptable salt thereof.

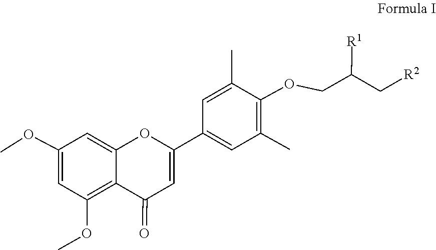

Certain aspects are directed to a compound of Formula I where R¹ is —H, —OH, halogen, alkoxy, —NH₂, or —CF₃; and R² is a substituted or unsubstituted cycloalkyl, substituted or unsubstituted heterocycle, or substituted or unsubstituted aryl or heteroaryl. In certain aspects R¹ is hydrogen. In other aspects R² is selected from a 3-6 membered substituted or unsubstituted cycloalkyl, or substituted or unsubstituted heterocycle having 1, 2, or 3 heteroatoms. In certain aspects the heteroatoms are oxygen, nitrogen, sulfur or combinations thereof. In certain aspects the heteroatom is nitrogen. In particular aspects the heterocycle is a piperazine.

Certain aspects a compound of Formula I is ZL0969 having a structure of:

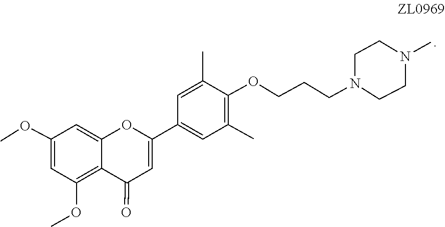

Chemical Definitions: Various chemical definitions related to such compounds are provided as follows.

As used herein, the term “nitro” means —NO₂; the term “halo” or “halogen” designates —F, —Cl, —Br or —I; the term “mercapto” means —SH; the term “cyano” means —CN; the term “azido” means —N₃; the term “silyl” means —SiH₃; the term “—OTs” means an O-tosyl group; and the term “hydroxyl” means —OH.

The term “alkyl,” by itself or as part of another substituent, means, unless otherwise stated, a linear (i.e., unbranched) or branched carbon chain, which may be fully saturated, mono- or polyunsaturated. An unsaturated alkyl group is one having one or more double bonds or triple bonds. Saturated alkyl groups include those having one or more carbon-carbon double bonds (alkenyl) and those having one or more carbon-carbon triple bonds (alkynyl). The groups, —CH₃ (Me), —CH₂CH₃ (Et), —CH₂CH₂CH₃ (n-Pr), —CH(CH₃)₂ (iso-Pr), —CH₂CH₂CH₂CH₃ (n-Bu), —CH(CH₃) CH₂CH₃ (sec-butyl), —CH₂CH(CH₃)₂ (iso-butyl), —C(CH₃)₃ (tert-butyl), —CH₂C (CH₃)₃ (neo-pentyl), are all non-limiting examples of alkyl groups.

The term “heteroalkyl,” by itself or in combination with another term, means, unless otherwise stated, a linear or branched chain having at least one carbon atom and at least one heteroatom selected from the group consisting of O, N, S, P, and Si. In certain embodiments, the heteroatoms are selected from the group consisting of O and N. The heteroatom(s) may be placed at any interior position of the heteroalkyl group or at the position at which the alkyl group is attached to the remainder of the molecule. Up to two heteroatoms may be consecutive. The following groups are all non-limiting examples of heteroalkyl groups: trifluoromethyl, —CH₂F, —CH₂ Cl, —CH₂ Br, —CH₂ OH, —CH₂ OCH₃, —CH₂ OCH₂ CF₃, —CH₂OC(O)CH₃, —CH₂ NH₂, —CH₂ NHCH₃, —CH₂ N(CH₃)₂, —CH₂CH₂Cl, —CH₂CH₂OH, CH₂CH₂OC(O)CH₃, —CH₂CH₂ NHCO₂C(CH₃)₃, and —CH₂ Si(CH₃)₃.

The terms “cycloalkyl” and “heterocycle,” by themselves or in combination with other terms, means cyclic versions of “alkyl” and “heteroalkyl”, respectively. Additionally, for heterocycle, a heteroatom can occupy the position at which the heterocycle is attached to the remainder of the molecule. Non-limiting examples of a heterocycle include piperazine; piperidine; pyridine; pyridazine; pyrimidine; pyrazine; 1,2,4-triazine; morpholine; or thiomorpholine.

The term “aryl” means a polyunsaturated, aromatic, hydrocarbon substituent. Aryl groups can be monocyclic or polycyclic (e.g., 2 to 3 rings that are fused together or linked covalently). The term “heteroaryl” refers to an aryl group that contains one to four heteroatoms selected from N, O, and S. A heteroaryl group can be attached to the remainder of the molecule through a carbon or heteroatom. Non-limiting examples of aryl and heteroaryl groups include phenyl, piperidyl, piperazinyl, morpholinyl, tetrahydropyranyl, tetrahydrofuranyl, pyrrolidinyl, dithianyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

The term “arylalkyl”, used alone or in any combination, refers to an aryl group which may be unsubstituted or substituted as previously defined and which is appended to the parent molecular moiety through an alkyl group.

Various groups are described herein as substituted or unsubstituted (i.e., optionally substituted). Optionally substituted groups may include one or more substituents independently selected from: halogen, nitro, cyano, hydroxy, amino, mercapto, formyl, carboxy, oxo, carbamoyl, substituted or unsubstituted alkyl, substituted or unsubstituted heteroalkyl, alkoxy, alkylthio, alkylamino, (alkyl)₂amino, alkylsulfinyl, alkylsulfonyl, arylsulfonyl, substituted or unsubstituted cycloalkyl, substituted or unsubstituted heterocyclyl, substituted or unsubstituted aryl, and substituted or unsubstituted heteroaryl. In certain aspects the optional substituents may be further substituted with one or more substituents independently selected from: halogen, nitro, cyano, hydroxy, amino, mercapto, formyl, carboxy, carbamoyl, unsubstituted alkyl, unsubstituted heteroalkyl, alkoxy, alkylthio, alkylamino, (alkyl)₂amino, alkylsulfinyl, alkylsulfonyl, arylsulfonyl, unsubstituted cycloalkyl, unsubstituted heterocyclyl, unsubstituted aryl, or unsubstituted heteroaryl. Examples of optional substituents include, but are not limited to:—OH, oxo (═O), —Cl, —F, Br, C₁₋₄alkyl, phenyl, benzyl, —NH₂, —NH(C₁₋₄alkyl), —N(C₁₋₄alkyl)₂, —NO₂, —S(C₁₋₄alkyl), —SO₂(C₁₋₄alkyl), —CO₂(C₁₋₄alkyl), and —O (C₁₋₄alkyl).

The term “ester” as used herein is defined refers to the general formula EOOC; wherein E is alkyl or aryl, as including a group of formula —COOR.

The term “alkoxy” means a group having the structure —OR′, where R′ is an optionally substituted alkyl or cycloalkyl group. The term “heteroalkoxy” similarly means a group having the structure-OR, where R is a heteroalkyl or heterocycle.

The term “amino” means a group having the structure-NR′R″, where R′ and R″ are independently hydrogen or an optionally substituted alkyl, heteroalkyl, cycloalkyl, or heterocyclyl group. The term “amino” includes primary, secondary, and tertiary amines.

The term “oxo” as used herein means an oxygen that is double bonded to a carbon atom.

The term “hydroxyalkyl” as used herein refers to hydroxylated straight or branched chain radicals containing one to ten carbon atoms, as illustrated by, but not limited to 2-propanol, 3-propanol and the like.

The term “alkylamino” as used herein includes mono- and dialkylamino groups, wherein an alkyl group contains from 1 to 6 carbon atoms which may be straight-chained or branched. Typical examples are methylamino, methylethylamino, diethylamino, propylamino, diisopropylamino, and hexylamino.

The term “alkylsulfonyl” as used herein means a moiety having the formula —S(O₂)—R′, where R′ is an alkyl group. R′ may have a specified number of carbons (e.g. “C₁₋₄ alkylsulfonyl”)

The term “pharmaceutically acceptable salts,” as used herein, refers to salts of compounds of this invention that are substantially non-toxic to living organisms. Typical pharmaceutically acceptable salts include those salts prepared by reaction of a compound of this invention with an inorganic or organic acid, or an organic base, depending on the substituents present on the compounds of the invention.

Non-limiting examples of inorganic acids which may be used to prepare pharmaceutically acceptable salts include: hydrochloric acid, phosphoric acid, sulfuric acid, hydrobromic acid, hydroiodic acid, phosphorous acid and the like. Examples of organic acids which may be used to prepare pharmaceutically acceptable salts include: aliphatic mono- and dicarboxylic acids, such as oxalic acid, carbonic acid, citric acid, succinic acid, phenyl-heteroatom-substituted alkanoic acids, aliphatic and aromatic sulfuric acids and the like. Pharmaceutically acceptable salts prepared from inorganic or organic acids thus include hydrochloride, hydrobromide, nitrate, sulfate, pyrosulfate, bisulfate, sulfite, bisulfate, phosphate, monohydrogenphosphate, dihydrogenphosphate, metaphosphate, pyrophosphate, hydroiodide, hydro fluoride, acetate, propionate, formate, oxalate, citrate, lactate, p-toluenesulfonate, methanesulfonate, maleate, and the like.

Suitable pharmaceutically acceptable salts may also be formed by reacting the agents of the invention with an organic base such as methylamine, ethylamine, ethanolamine, lysine, ornithine and the like. Pharmaceutically acceptable salts include the salts formed between carboxylate or sulfonate groups found on some of the compounds of this invention and inorganic cations, such as sodium, potassium, ammonium, or calcium, or such organic cations as isopropylammonium, trimethylammonium, tetramethylammonium, and imidazolium.

It should be recognized that the particular anion or cation forming a part of any salt of this invention is not critical, so long as the salt, as a whole, is pharmacologically acceptable.

Additional examples of pharmaceutically acceptable salts and their methods of preparation and use are presented in Handbook of Pharmaceutical Salts: Properties, Selection and Use (2002), which is incorporated herein by reference.

It is contemplated that any embodiment discussed in this specification can be implemented with respect to any method or composition of the invention, and vice versa. Furthermore, compositions of the invention can be used to achieve methods of the invention.

### II. ANTI-INFLAMMATORY AGENTS

In certain aspects of the invention an anti-inflammatory agent may be used in combination with a composition described herein. The anti-inflammatory can be a steroidal or non-steroidal anti-inflammatory.

Steroidal anti-inflammatoires for use herein include, but are not limited to fluticasone, beclomethasone, any pharmaceutically acceptable derivative thereof, and any combination thereof. As used herein, a pharmaceutically acceptable derivative includes any salt, ester, enol ether, enol ester, acid, base, solvate or hydrate thereof. Such derivatives may be prepared by those of skill in the art using known methods for such derivatization.

Fluticasone-Fluticasone propionate is a synthetic corticosteroid. Fluticasone propionate is a white to off-white powder and is practically insoluble in water, freely soluble in dimethyl sulfoxide and dimethylformamide, and slightly soluble in methanol and 95% ethanol. In an embodiment, the formulations of the present invention may comprise a steroidal anti-inflammatory (e.g., fluticasone propionate).

Beclomethasone—In certain aspects the steroidal anti-inflammatory can be beclomethasone dipropionate or its monohydrate. The compound may be a white powder and is very slightly soluble in water (Physicians' Desk Reference), very soluble in chloroform, and freely soluble in acetone and in alcohol.

Providing steroidal anti-inflammatoires according to the present invention may enhance the compositions and methods of the invention by, for example, attenuating any unwanted inflammation. Examples of other steroidal anti-inflammatoires for use herein include, but are not limited to, betamethasone, triamcinolone, dexamethasone, prednisone, mometasone, flunisolide and budesonide.

In accordance with yet another aspect of the invention, the non-steroidal anti-inflammatory agent may include aspirin, sodium salicylate, acetaminophen, phenacetin, ibuprofen, ketoprofen, indomethacin, flurbiprofen, diclofenac, naproxen, piroxicam, tebufelone, etodolac, nabumetone, tenidap, alcofenac, antipyrine, amimopyrine, dipyrone, ammopyrone, phenylbutazone, clofezone, oxyphenbutazone, prexazone, apazone, benzydamine, bucolome, cinchopen, clonixin, ditrazol, epirizole, fenoprofen, floctafeninl, flufenamic acid, glaphenine, indoprofen, meclofenamic acid, mefenamic acid, niflumic acid, salidifamides, sulindac, suprofen, tolmetin, nabumetone, tiaramide, proquazone, bufexamac, flumizole, tinoridine, timegadine, dapsone, diflunisal, benorylate, fosfosal, fenclofenac, etodolac, fentiazac, tilomisole, carprofen, fenbufen, oxaprozin, tiaprofenic acid, pirprofen, feprazone, piroxicam, sudoxicam, isoxicam, celecoxib, Vioxx®, and/or tenoxicam.

### III. ANTIVIRAL AGENTS

In certain aspects of the invention an anti-viral agent may be used in combination with a composition described herein. Anti-viral agents include, but are not limited to nucleoside analogs, antisense RNA, or monoclonal antibodies (see for example Hruska et al., 1980, Antimicrobial Agents and Chemotherapy 17:770-75; Leaman et al., 2002, Virology (New York NY) 292:70-77; Lai et al., 2008, Mol Ther. 16:1120-28).

Antiviral agents herein include but are not limited to synthetic ribonucleosides (ribavirin), anti-sense oligonucleotides, interfering nucleic acids, neutralizing antibodies, interferons or any combination thereof. As used herein, a pharmaceutically acceptable derivative includes any salt, ester, enol ether, enol ester, acid, base, solvate or hydrate thereof. Nucleotides may also be encapsulated into liposomes for more efficient intracellular transfer. These compounds may be administered systemically, via aerosol, or nebulization. Such derivatives may be prepared by those of skill in the art using known methods for such derivatization.

Ribavirin is a nucleoside analog with broad-spectrum inhibitory activities towards RNA viruses used in severe RSV and hemorrhagic fever infections.

Antisense oligonucleotides and modified nucleic acids, such as morpholino oligonucleotides, targeted towards RSV genomic RNA have been used to reduce viral replication.

Humanized monoclonal antibodies (palivizumab, Synagis) raised against RSV structural proteins have been used for the prophylaxis and treatment of patients at high risk for RSV disease.

Interferons-interferons are immune regulating hormones that limit viral replication. These can be used alone or in combination with ribavirin to enhance anti-viral immunity.

### IV. FORMULATION AND ADMINISTRATION

Certain aspects are directed to methods of treating a subject with, expected of having, or has been exposed to a virus, e.g., coronavirus, comprising administering an effective amount of a BRD4 inhibitor, such as the compounds described herein. The method can further include administering an anti-inflammatory compound. The virus can be a respiratory virus, and in particular a respiratory syncytial virus (RSV). The methods can further include administering an anti-viral compound.

Compound(s) described herein or a pharmaceutically acceptable salt thereof, may be used in combination with one or more additional therapeutic agents or treatments which act by the same mechanism or by different mechanisms to treat a disease. The different therapeutic agents or treatments may be administered sequentially or simultaneously, in separate compositions or in the same composition. Therapeutical agents that may be used in combinations include, but are not limited to cidofovir triphosphate, cidofovir, abacavir, ganciclovir, stavudine triphosphate, 2′-O-methylated UTP, desidustat, ampion, trans sodium crocetinate, CT-P59, Ab8, heparin, apixaban, GC373, GC376, Oleandrin, GS-441524, sertraline, Lanadelumab, zilucoplan, abatacept, CLBS119, Ranitidine, Risankizumab, AR-711, AR-701, MP0423, bempegaldesleukin, melatonin, carvedilol, mercaptopurine, paroxetine, casirivimab, imdevimab, ADG20, emricasan, dapansutrile, ceniciviroc infliximab, DWRX2003, AZD7442, MAN-19, LAU-7b, niclosamide, ANA001, fluvoxamine, narsoplimab, Sarconcos, GIGA-2050, VERU-111, REGN-COV2, icatibant, cenicriviroc, NTR-441, LAM-002A, oseltamivir, VHH72-Fc, MK-4482, EB05, OB-002, CM-4620-IE, IMU-838, SNG001, NT-17, BOLD-100, WP1122, itolizumab, PB1046, fostamatinib, colchicine, M5049, EDP1815, ABX464, CPI-006, azclastine, garadacimab, silmitasertib, lopinavir, ritonavir, remdesivir, cloroquine, hydrochloroquine, convalescent plasma transfusion, azithromycin, tocilizumab, famotidine, sarilumab, interferon beta, interferon beta-la, interferon beta-1b, peginterferon lambda-la, favipiravir, ASDC-09, dapagliflozin, CD24Fc, ribavirin, umifenovir, nitric oxide, APN01, teicoplanin, oritavancin, dalbavancin, monensin, ivermectin, darunavir, cobicistat, fingolimod, camostat, galidesicir, thalomide, leronlimab, remestemcel-L, canakinumab, TAK-888, azvudine, BPI-002, AT-100, T-89, Neumifil, GreMERSfi, liposomal curcumin, OYA-1, oxypurinol, mosedipimod, PUL-042, naltrexone, metenkefalin, COVID-EIG, TNX-1800, ATR-002, 177Lu-EC-Amifostine, 99mTc-EC-Amifostine, apabetalone, STI-6991, STI-4398, antroquinonol, ZIP-1642, DPX-COVID-19, belapectin, GX-19, AdCOVID, siltuximab, IBIO-200, plitidepsin, C-21, meplazumab, pathogen-specific aAPC, LV-SMENP-DC, ARMS-I, rhu-pGSN, PRTX-007, CK-0802, namilumab, upamostat, NI-007, COVID-HIG, CYNK-001, Nafamostat, brilacidin, mavrilimumab, IPT-001, PittCo Vacc, allo-APZ2-Covid19, ENU-200, VIR-7832, VIR-7831, pritumumab, Ampion, TZLS-501, sodium pyruvate, silmitasertib, CoroFlu, BDB-1, AT-001, BLD-2660, 20-hydroxyecdysone, IFX-1, clsulfavirine, emapalumab, CEL-1000, trabedersen, VBI-2901, ASC-09, TJM-2, RPH-104, tranexamic acid, WP-1122, olokizumab, APN-01, danoprevir, piclidenoson, FW-1022, CORAVAX, Lamellasome COVID-19, COVID-19 XWG-03, EIDD-2801, AVM-0703, DC-661, acalabrutinib, bitespiramycin, Allocetra, tradipitant, bacTRL-Tri, Ad5-nCOV, EPV-CoV19, ADX-629, vazegepant, mercaptamine, sonlicromanol, aviptadil, fenretinide, IT-139, nitazoxanide, apabetalone, lucinactant, bacTRL-Spike, SAB-185, NVX-CoV2373, CM-4620, INO-4800, cicosapentaenoic acid, itanapraced, rintatolimod, XAV-19, niclosamide, ciclesonide, DAS181, ORBCEL-C, Metablok, dantrolene, CD24-IgFc, fadraciclib, gimsilumab, seliciclib, Cyto-MSC, ST-266, MRx-0004, ravulizumab, tafoxiparin, DAS-181, BMS-986253, cholecalciferol, nafamostat, ChAd0x1 nCOV-19, idronoxil, LY-3127804, ATYR-1923, VPM-1002, Mycobacterium w, lenzilumab, Polyoxidonium, conestat alfa, ubiquitin proteasome modulator, COVID-19 virus main protease Mpro inhibitor, mRNA-1273, clevudine, bucillamine, sodium meta-arsenite, vidofludimus, DARPin, COV-ENT-1, KTH-222, mefuparib, brensocatib, zanubrutinib, anakinra, selinexor, sarilumab, astodrimer, dapagliflozin propanediol, opaganib, BNT-162c2, BNT-162b2, BNT-162b1, BNT-162al, ifenprodil, PIC1-01, 2X-121, zotatifin, aplidin, cloperastine, clemastine, dociparstat, avdoralimab, VIR-2703, ALN-COV, intravenous immunoglobulin (IVIg), apremilast, vicromax, baloxavir marboxil, emtricitabine, tenofovir, novaferon, secukinumab, valsartan, imatinib, omalizumab, leucine, sofosbuvir, alovudine, zidovudine, R-107, AB-201, sargramostim, LYT-100, senicapoc, fluvoxamine, aspirin, losartan, ADX-1612, ADX-629, sirikumab, otilimab, STI-1499, TR-C19, ABX-464, interferon alpha2b, arbidol, 5309, vafidemstat, AT-527, ibudilast, auxora, bemcentinib, eculizumab, JS016, FSD-201, LY-CoV555, avifavir, OP-101, RLF-100, DMX-200, 47D11, remsima, TYR1923, dexamethasone, EDP-1815, PTC29, rabeximod, foralumab, budesonide, molnupiravir, ensovibep, dalcetrapib, FSD201, pralatrexate, proxalutamide, clofazimine and merimepodib.

In some embodiments, compounds of Formula I, or a pharmaceutically acceptable salt thereof, is used in combination with an antiviral. In some embodiments, compound 1, or a pharmaceutically acceptable salt thereof, is used in combination with a corticosteroid. In some embodiments, compound 1, or a pharmaceutically acceptable salt thereof, is used in combination with an antiviral and a corticosteroid. In some embodiments, the antiviral is remdesivir. In some embodiments, the antiviral is favipiravir. In some embodiments, the corticosteroid is dexamethasone.

Also provided, herein, is a pharmaceutical composition comprising a compound of Formula I, or a pharmaceutically acceptable salt thereof, and one or more other therapeutic agents. The therapeutic agent may be selected from the class of agents specified above and from the list of specific agents described above. In some embodiments, the pharmaceutical composition is suitable for delivery to the lungs. In some embodiments, the pharmaceutical composition is suitable for inhaled or nebulized administration. In some embodiments, the pharmaceutical composition is a dry powder or a liquid composition.

Further, for all the methods disclosed herein, the methods comprise administering to the mammal, human or patient, a compound of Formula I, or a pharmaceutically acceptable salt thereof, and one or more other therapeutic agents.

When used in combination therapy, the agents may be formulated in a single pharmaceutical composition, or the agents may be provided in separate compositions that are administered simultaneously or at separate times, by the same or by different routes of administration. Such compositions can be packaged separately or may be packaged together as a kit. The two or more therapeutic agents in the kit may be administered by the same route of administration or by different routes of administration.

The compounds and pharmaceutical compositions disclosed herein may be administered, for example, via the respiratory system of a subject. In certain aspects the compositions are deposited in the lung by methods and devices known in the art. Therapeutic compositions described herein may be prepared in water suitably mixed with a surfactant, such as hydroxypropylcellulose. Dispersions may also be prepared in glycerol, liquid polyethylene glycols and mixtures thereof, and in oils. Under ordinary conditions of storage and use, these preparations contain a preservative to prevent the growth of microorganisms. In certain aspects the compounds described herein can be formulated for extended release as a nanoparticles (NPs) formulation, made from biodegradable and biocompatible polymers. Such therapeutic formulations offer a platform for reducing the number of doses, reduce toxicity without altering its therapeutic effects, protect the drug from inactivation (due to protein binding or metabolism of the drug), and provide a sustained release stable for long periods of time and have greater specificity against target tissues (given by the functionalization of the molecule).

The pharmaceutical forms suitable for inhalation include sterile aqueous solutions or dispersions and sterile powders for the extemporaneous preparation of sterile inhalable solutions or dispersions. In all cases the form is typically sterile and capable of inhalation directly or through some intermediary process or device. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. The prevention of the action of microorganisms can be brought about by various antibacterial and antifungal agents, for example, parabens, chlorobutanol, phenol, sorbic acid, thimerosal, and the like.

Some variation in dosage will necessarily occur depending on the condition of the subject being treated and the circumstances involving exposure or potential exposure. The person responsible for administration will, in any event, determine the appropriate dose for the individual subject. Moreover, for human administration, preparations should meet sterility, pyrogenicity, general safety, and purity standards as required by FDA Office of Biologics standards or other similar organizations.

Sterile compositions are prepared by incorporating the active components in the required amount in the appropriate solvent with various other ingredients enumerated above, as required, followed by, for example, filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile compositions, some methods of preparation are vacuum-drying and freeze-drying techniques which yield a powder of the component(s) and/or active ingredient(s) plus any additional desired ingredient from a previously sterile-filtered solution.

In certain embodiments the compounds can be associated with the surface of, directly or indirectly conjugated to, encapsulated within, surrounded by, dissolved in, or dispersed throughout a polymeric matrix. The phrase “loaded into”, “loaded onto”, “incorporated into”, or “included in” are used interchangeably to generally describe the association of the compound with the particle without imparting any further meaning as to where or how the compound is associated with the particle.

The amount of compound present in a particle (entrapment efficiency) can be at least about 10% to as high as about 98% w/w. In some embodiments, the entrapment efficiency can be about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 98% (w/w).

Pulmonary/respiratory drug delivery can be implemented by different approaches, including liquid nebulizers, aerosol-based metered dose inhalers (MDI's), sprayers, dry powder dispersion devices and the like. Such methods and compositions are well known to those of skill in the art, as indicated by U.S. Pat. Nos. 6,797,258; 6,794,357; 6,737,045; and 6,488,953-all of which are incorporated by reference. According to the invention, at least one pharmaceutical composition can be delivered by any of a variety of inhalation or nasal devices known in the art for administration of a therapeutic agent by inhalation. Other devices suitable for directing pulmonary or nasal administration are also known in the art. Typically, for pulmonary administration, at least one pharmaceutical composition is delivered in a particle size effective for reaching the lower airways of the lung or sinuses. Some specific examples of commercially available inhalation devices suitable for the practice of this invention are Turbohaler™ (Astra), Rotahaler®) (Glaxo), Diskus® (Glaxo), Spiros™ inhaler (Dura), devices marketed by Inhale Therapeutics, AERx™ (Aradigm), the Ultravent® nebulizer (Mallinckrodt), the Acorn II® nebulizer (Marquest Medical Products), the Ventolin® metered dose inhaler (Glaxo), the Spinhaler® powder inhaler (Fisons), Acrotech II® or the like.

All such inhalation devices can be used for the administration of a pharmaceutical composition in an aerosol. Such aerosols may comprise either solutions (both aqueous and non-aqueous) or solid particles. Metered dose inhalers typically use a propellant gas and require actuation during inspiration. Sec, e.g., WO 98/35888 and WO 94/16970. Dry powder inhalers use breath-actuation of a mixed powder. Sec U.S. Pat. Nos. 5,458,135 and 4,668,218; PCT publications WO 97/25086, WO 94/08552 and WO 94/06498; and European application EP 0237507, each of which is incorporated herein by reference in their entirety. Nebulizers produce aerosols from solutions, while metered dose inhalers, dry powder inhalers, and the like generate small particle aerosols. Suitable formulations for administration include, but are not limited to nasal spray or nasal drops, and may include aqueous or oily solutions of a composition described herein.

A spray comprising a pharmaceutical composition described herein can be produced by forcing a suspension or solution of a composition through a nozzle under pressure. The nozzle size and configuration, the applied pressure, and the liquid feed rate can be chosen to achieve the desired output and particle size. An electrospray can be produced, for example, by an electric field in connection with a capillary or nozzle feed.

A pharmaceutical composition described herein can be administered by a nebulizer such as a jet nebulizer or an ultrasonic nebulizer.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically acceptable” refers to molecular entities and compositions that do not produce an allergic or similar untoward reaction when administered to a subject. The preparation of an aqueous composition that contains a polypeptide or peptide as an active ingredient is well understood in the art.

### V. KITS AND DEVICES

The invention provides a variety of kits for conveniently and/or effectively carrying out methods of the present invention. Typically, kits will comprise a sufficient amount and/or numbers of components to allow a user to perform multiple treatments of a subject(s) and/or to perform multiple experiments.

In one aspect, the present invention provides kits comprising one or more compound of the invention.

The kit may further comprise packaging and instructions and/or a delivery agent to form a formulation composition. The delivery agent may comprise a saline, a buffered solution, or a delivery agent.

In one embodiment, the buffer solution may include sodium chloride, calcium chloride, phosphate and/or EDTA. In another embodiment, the buffer solution may include, but is not limited to, saline, saline with 2 mM calcium, 5% sucrose, 5% sucrose with 2 mM calcium, 5% Mannitol, 5% Mannitol with 2 mM calcium, Ringer's lactate, sodium chloride, sodium chloride with 2 mM calcium and mannose. In a further embodiment, the buffer solutions may be precipitated or it may be lyophilized. The amount of each component may be varied to enable consistent, reproducible higher concentration saline or simple buffer formulations. The components may also be varied to increase the stability of compound(s) in the buffer solution over a period of time and/or under a variety of conditions.

Devices. The present invention provides for devices which may incorporate one or more compound of the invention.

Devices for administration may be employed to deliver one or more compound of the invention of the present invention according to single, multi- or split-dosing regimens taught herein.

### VI. EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Decreasing Viral Titer in Mouse Model of Covid19

High levels of CXCL10 expression in infected patients correspond to high risk of unfavorable outcome (ICU admission or death) (Lorè et al. Mol Med 27:129, 2021; Gudowska-Sawczuk and Mroczko, Int J Mol Sci 23 (7): 3673, 2022; Thwaites et al. Sciimmunol. 6:57, 2021). BRD4 is a novel histone acetyltransferase (HAT) that acetylates histones H3 and H4, and contributes to the pathology of cytokine storms.

### A. RESULTS

Because SARS-COV-2 generates an inflammatory response in the lungs that may result in acute formation of lung injuries, pneumonia, and death the inventors chose human lung cell monolayers (Calu-3) to screen a panel of thirty-one epigenetic inhibitors for potential effects on fold-changes in expression of CXCL10 following infection with SARS-COV-2. BRD4 inhibitor, ZL0969, reduces expression levels of CXCL10 ameliorating CXCL10 function in inflammatory response in lungs.

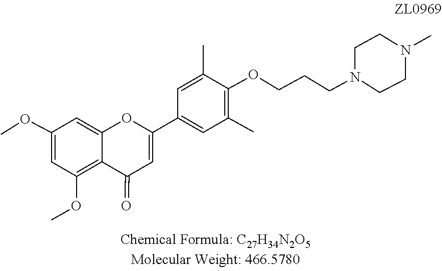

### B. MATERIALS AND METHODS

All work with SARS-COV-2 was performed in biosafety level 3 (BSL-3) and biosafety level 4 (BSL-4) facilities of the Galveston National Laboratory. RNA purification, cDNA preparation, qPCR, histopathology, and Next Generation sequencing were performed using samples that were inactivated and removed from BSL-3 and BSL-4 containment facilities according to University of Texas Medical Branch (UTMB) approved standard operating procedures (SOP).

Tissue culture and virus propagation. Calu-3 cells (ATCC) were maintained in Eagle's Minimum Essential Medium (Gibco) supplemented with 20% Fetal Bovine Serum (FBS) (Bio-Technc) and 1% Penicillin-Streptomycin (10,000 U/mL) (Gibco). Vero-E6 cells (ATCC) were maintained in Eagle's Minimum Essential Medium (Gibco) supplemented with 10% FBS (Bio-Techne), 1% sodium pyruvate (Sigma), 1% Non-Essential Amino Acids (Sigma) and 0.1% gentamicin sulfate (Corning). All cells were grown at 37° C. and 5% CO₂.

rSARS-COV-2 expressing Neon Green protein (mNG) propagated in Vero-E6 cells. Titers were determined by plaque assay to detect fluorescent foci using wide-field fluorescence microscope (Olympus), (limit of detection, 7 FFU/mL). SARS-COV-2, B.1.351-Beta strain was kindly provided by Dr. Kenneth Plante with the UTMB World Reference Collection and propagated in Vero-E6 cells. Viral titer was determined by plaque assays in 96-well plates. Plates were incubated 2 days at 37° C. before being fixed with 10% formalin (ThermoFisher Scientific) and removed from containment per UTMB SOP. Fixed monolayers were stained with 10% formalin containing 0.25% crystal violet (ThermoFisher Scientific) at room temperature for 10 min and washed with water. Plaques were counted under microscope (Olympus), (limit of detection, 7 PFU/mL). Work with strains of SARS-COV-2 was done under biosafety level 3 (BSL-3) and biosafety level 4 (BSL-4) conditions at the Galveston National Laboratory. Virus inactivation was performed according to UTMB SOP.

rSARS-COV-2mNG infection of human lung cells and treatment with epigenetic inhibitors. Thirty-one epigenetic inhibitors were reconstituted in DMSO (Sigma). Calu-3 cells (4×10⁵ cells/mL) were seeded at 2×10⁵ cells per well onto the apical chambers of transwell 12 well plates (Costar) and incubated for 4-6 hours after which 1 mL fresh media was added to the basolateral chamber. Cells were incubated for 5 days to achieve confluency. On the day of infection, media was carefully aspirated from both chambers and fresh media containing 10 μM drug was added to the basolateral (1 mL) and the apical chamber (100 μL) chamber at least one hour prior to infection. Plates were transferred to the BSL-4 laboratory where media was carefully aspirated from both chambers and cells were infected with 100 μL rSARS-COV-2mNG at MOI of 0.3 FFU per cell applied directly onto the cell monolayer in the apical chamber. After one-hour incubation with virus, cell monolayers were washed with PBS (Corning) and fresh media containing 10 μM drug was added once again to the basolateral (1 mL) and apical (100 μL) chambers. After overnight incubation, the media in the basolateral chamber was aspirated and 1 mL fresh media containing 10 μM drug was added to the basolateral chamber. The second day after infection, cell monolayers were collected and inactivated in TRIzol (Invitrogen) and removed from the BSL-4 containment facility per UTMB SOP.

Epigenetic inhibitor screening for changes in CXCL10 expression. Total RNA from infected Calu-3 cell monolayers was purified using the Direct-zol RNA Microprep kit (Zymo) and quantitated with the NanoDrop 2000 Spectrophotometer. cDNA was prepared by following the Invitrogen Life Technologies Protocol Pub No. MAN0013443 Rev. A.O, Superscript IV Reverse Transcriptase User Guide, using 200 ng template RNA. Real-time PCR assays were prepared following the TaqMan Fast Advanced Master Mix User Guide (Publication Number 4444605, Revision D) by adding 4 ng cDNA to 5 μL TaqMan Fast Advanced Master Mix (ThermoFisher Scientific), 0.5 L PrimeTime Std qPCR Assay ACTB (Probe 5′-/5Cy5/TCATCCATGGTGAGCTGGCGG/31AbRQSp/-3′ (SEQ ID NO:1), Primer 1-5′-ACAGAGCCTCGCCTTTG-3′, Primer 2-5′-CCTTGCACATGCCGGAG-3′ (SEQ ID NO:2)), and 0.5 pL PrimeTime Std qPCR Assay CXCL10 (Probe 5′-/56-FAM/ACCTCCAGT/ZEN/CTCAGCACCATGAATC/3IABKFQ/-3′ (SEQ ID NO:3), Primer 1-5′-GACATATTCTGAGCCTACAGCA-3′ (SEQ ID NO:4), Primer 2-5′-CAGTTCTAGAGAGAGGTACTCCT-3′ (SEQ ID NO:5)) (Integrated DNA Technologies).

Reactions were assembled in MicroAmp Optical 96-well Reaction Plates (Applied Biosystems) and run using the QuantStudio 6 Real-Time PCR System (Applied Biosystems) under the following reaction conditions: 50° C. for 2 minutes and 95° C. for 2 minutes followed by 40 cycles denaturing at 95° C. for Is and annealing/extension at 60° C. for 20 s. Analysis was performed using QuantStudio Real Time PCR Software v.1.3.

Cytokine expression array of treated Calu-3 cells infected with SARS-COV-2mNG. cDNA from samples of infected and mock-infected Calu-3 cells treated with one of three of the inhibitors screened above (JMX0281, JMX0312, and ZL0969) or mock-treated, were screened using the TaqMan Array Human Cytokine Network, Fast 96-well TaqMan Gene Expression Assay (Applied Biosystems) for changes in cytokine expression using the TaqMan Gene Expression Assays User Guide, Publication 4391016, Revision K. Ten nanograms cDNA prepared in the section above was added to 5 μL TaqMan Fast Advanced Master Mix in each well of the array plate. Plates were run using the QuantStudio 6 Real-Time PCR System (Applied Biosystems) under the following reaction conditions: UNG incubation 50° C. for 2 minutes and Enzyme activation 95° C. for 20 s followed by 40 cycles denaturing at 95° C. for Is and annealing/extension at 60° C. for 20 s. Analysis was performed using QuantStudio Real Time PCR Software v.1.3.

In vivo mouse study. SARS-COV-2 infections of mice were performed in the animal BSL-3 (ABSL-3) containment facility at the Galveston National Laboratory. The animal protocols for testing epigenetic inhibitors in mice were approved by the Institutional Animal Care and Use Committee at UTMB. Seven-to eight-week-old wild-type male BALB/cJ mice were obtained from The Jackson Laboratory (catalog no. 00651). Four groups of mice (n=10) were infected with 6×10⁴ PFU SARS-COV-2, B.1.351-Beta strain via intranasal route and one group of mice (n=5) was mock-infected with PBS and mock-treated with vehicle via the same route. Three mice did not survive challenge (1 JMX0281 treated, 1 ZL0969 treated, and 1 mock-treated plus virus). The virus stock was back titrated at the time of infection to verify virus titer. Inhibitor stocks were prepared at a concentration of 200 mg/mL in DMSO and kept frozen. Fresh daily treatments were prepared to a final concentration of 2 mg/mL in PBS (ZL0969) or 60% PEG400 in PBS (JMX0281 and JMX0312). Mice received daily administration of 200 μL drug or placebo (vehicle) via intraperitoneal route beginning 2 days prior to challenge with virus and continuing through day 5 post-challenge. Animals were monitored daily for weight loss and signs of disease. There were two euthanasia events. Twenty three treated mice were euthanized on day 2 post-challenge for tissue harvest (13 treated mice, 5 mock-treated mice, and 5 uninfected mice). The remaining 19 mice were euthanized on day 10 post-challenge to monitor weight and health and for tissue harvest.

Viral load in superior and middle lobes of mouse lungs. Tissues from animals euthanized on day 2 and day 10 post-challenge were harvested in CryoELITE cryogenic vials (Wheaton) which were quickly sealed and frozen. Tissues were later transferred to tubes containing 1 mL PBS (Corning) and homogenized using the TissueLyser II system (Qiagen). Lung homogenates were titrated on Vero E6 cell monolayers in 96-well plates to determine viral loads. Plates were incubated 2 days at 37° C. before being fixed with 10% formalin (ThermoFisher Scientific) and removed from containment per UTMB SOP. Fixed monolayers were stained with 10% formalin containing 0.25% crystal violet (ThermoFisher Scientific) at room temperature for 10 min and washed with water. Plaques were counted under microscope (Olympus), and virus load per gram of tissue was determined.

Histopathology of inferior and post-caval lobe of mouse lungs. Tissues from animals euthanized on day 2 post-challenge were harvested, fixed, and inactivated in 10% formalin (ThermoFisher Scientific), then removed from containment per UTMB SOP. Five-micron sections were prepared and stained with hematoxylin and eosin. A histologic scoring system was used based upon known pathological characteristics of SARS-COV-2 infection. The scoring included the presence and extent of inflammatory infiltrates, edema, hyperemia and congestion, alveolar hemorrhages, degeneration of alveolar lining, necrosis, deposition of proteinaceous debris/membranous structure and endothelial swelling. A total of 20 fields of blinded tissue sections were examined under a light microscope (Olympus with cellSens imaging software) and semi-quantitatively scored for pathological lesions using the following criteria: 0 for no changes, 1 for mild changes, 2 for moderate changes, 3 for marked changes, and 4 for severe changes.

Next generation sequencing of RNA from left lobe of mouse lungs. Tissues from animals euthanized on day 2 post-challenge were harvested and stored in RNALater Solution (Invitrogen). Tissues were later transferred to tubes containing 1 mL TRIzol (Invitrogen), homogenized using the TissueLyser II system (Qiagen) and inactivated and removed from containment per UTMB SOP. Total RNA was purified using the Direct-zol RNA Miniprep kit (Zymo) and quantitated with the NanoDrop 2000 Spectrophotometer. RNA quality was deemed satisfactory using the Agilent Bioanalyzer 2100. An RNA-Seq Non-Stranded library was produced followed by sequencing on the NextSeq550 platform for 400 million reads. Data analysis was performed by the UTMB Next Generation Sequencing Core Facility.

Statistical analysis. Data is presented as mean and standard error for each treatment group at each time point. Comparison of viral load was conducted by two-way ANOVA and a post hoc analysis comparing groups pairwise by time point. Dunnett's test was perform for multiple testing correction. Significance was set at 0.05. Analysis was performed using GraphPad Prism (version 9.0.0) (GraphPad Software, Inc.).

1. A method of treating a patient infected with a coronavirus comprising administering to the patient a compound of Formula 1 or a pharmaceutically-acceptable salt thereof 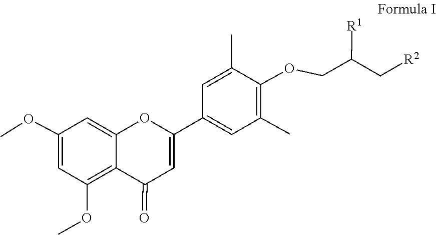
wherein R¹ is selected from hydrogen or hydroxyl; and
R² is selected from a substituted or unsubstituted cycloalkyl, or a substituted or unsubstituted heterocycle.

2. The method of claim 1, wherein R¹ is hydrogen and R² is a substituted or unsubstituted heterocycle.

3. The method of claim 2, wherein R² is a substituted heterocycle.

4. The method of claim 3, wherein the substituted heterocycle is a substituted piperazine.

5. The method of claim 4, wherein the substituted piperazine is a methyl piperazine.

6. The method of claim 5, wherein the methyl piperazine is a 4 methyl piperazine.

7. The method of claim 1, wherein the coronavirus is selected from the group consisting of SARS-COV, SARS-COV-2, and MERS-COV.

8. The method of claim 1, wherein the coronavirus is SARS-COV-2.

9. The method of claim 1, wherein the compound, or a pharmaceutically-acceptable salt thereof, is administered by inhalation.

10. The method of claim 1, wherein the compound, or a pharmaceutically-acceptable salt thereof, is administered by nebulized inhalation.

11. The method of claim 1, wherein the compound, or a pharmaceutically-acceptable salt thereof, is administered once a day.

12. The method of claim 1, wherein the compound, or a pharmaceutically-acceptable salt thereof, is administered at a higher loading dose on day 1 of administration followed by a lower dose on the following days.

13. The method of claim 1, wherein the method comprises administering one or more additional therapeutic agents or treatments to the patient.

14. The method of claim 1, wherein the patient receives standard of care co-treatment.

15. The method of claim 1, wherein the patient is also treated with corticosteroids.

16. The method of claim 1, wherein the patient is also treated with dexamethasone.

17. The method of claim 1, wherein the patient is also treated with remdesivir.

18. The method of claim 1, wherein the compound of Formula 1, or a pharmaceutically-acceptable salt thereof, is administered to the patient at a dose of about 1 mg to about 10 mg.

19. The method of claim 1, wherein the patient has mild to moderate COVID-19.

20. The method of claim 1, wherein the patient has severe COVID-19.

21. The method of claim 1, wherein the patient is at high risk for progressing to severe COVID-19 and/or hospitalization.

22. The method of claim 1, wherein the patient suffers from hypertension and/or diabetes.
